SEQUENCE LISTING (5 sequences)

SEQ ID NO: 1 - DNA, 31 bp, Artificial sequence
  Synthetic sequence
cgccatatgagtcaatcgatctgttcaacag

SEQ ID NO: 2 - DNA, 42 bp, Artificial sequence
  Synthetic sequence
cgcggatccttattgttttttcgctttagaaggtaaaaaacc

SEQ ID NO: 3 - DNA, 564 bp, Escherichia coli
atgggccatcatcatcatcatcatcatcatcatcacagcagcggccatatcgacgacgacgacaagcatatgagtcaatcgatctgttcaacagggctacgctggctgtggctggtggtagtcgtgctgattatcgatctgggcagcaaatacctgatcctccagaactttgctctgggggatacggtcccgctgttcccgtcgcttaatctgcattatgcgcgtaactatggcgcggcgtttagtttccttgccgatagcggcggctggcagcgttggttctttgccggtattgcgattggtattagcgtgatcctggcagtgatgatgtatcgctcgaaggccacgcagaagctaaacaatatcgcttacgcgctgattattggcggcgcgctgggcaacctgttcgaccgcctgtggcacggcttcgttgtcgatatgatcgacttctacgtcggcggctggcacttcgccaccttcaaccttgccgatactgccatctgtgtcggtgcggcactgattgtgctggaaggttttttaccttctaaagcgaaaaaacaataa

SEQ ID NO: 4 - DNA, 483 bp, Streptococcus pyogenes
atgaaaaaacgattgtttgtgcttagcttgatcctccttgtagctttggatcaacttagtaaattttggattgtttctcatatagcgcttggagaagtgaaaccctttatcccaggtatcgtcagcttgacttacttgcaaaacaatggggctgccttttccatattgcaggaccagcaatggttctttgttgtcataacggttttagttatcggttatgctatttattaccttgctactcatccccatttaaatatctggaaacaattagctctcttgcttattatttctggtggaatcgggaattttattgatcgtttgcgtttagcttacgtgattgatatgattcatttagactttgtggattttgccatttttaatgtggcagattcataccttaccgttggtgtcatattattattgatatgtttatggaaagaagaggattatggaaatctcgagcaccaccaccaccaccactga

SEQ ID NO: 5 - DNA, 630 bp, Thermotoga maritima
atgggccatcatcatcatcatcatcatcatcatcacagcagcggccatatcgacgacgacgacaagcatatggcgtttgtgatggttctcacaattgttctggatcagcttacaaagcggatagcaagcgagatacacggaacttttttcatagttccgggttttttgagattcgtgaaggcaaccaaccgaggaatcgcactcgggttgtttaaaaatctttccgaacagcttctctggaccgtgatgttcgttgttgtttttctctccctgcttccttatattttcaagttcagcaggctggaaagaatagccatgggcttcattcttgggggagctctcggcaaccttctcgacagaatcagattcggatacgttcttgattttctgaacttgacctttctcccaacgatattcaacctagcggatgtgttcatcatagtcggaggagcgcttatgatactgggagttttcagaggtggagacaatgaaagtttggagagtcgaaaaaagagaagagggctggagactggatcagtttttgaaggagaagacaccatcatggatctcgagatcaatgattcaaaaagcgataaaagagggcaaagtgaaggtcaacggtcagattaa

(19) United States
(12) Patent Application Publication
(10) Pub. No.: US 2019/0144912 A1
(43) Pub. Date: May 16, 2019

(54) METHODS FOR IDENTIFYING NOVEL ANTIBIOTICS AND RELATED COMPOSITIONS

(71) Applicant: THE SCRIPPS RESEARCH INSTITUTE, La Jolla, CA (US)

(72) Inventors: Dennis Wolan, La Jolla, CA (US); Anna Owensby, San Diego, CA (US)

(21) Appl. No.: 15/756,741

(22) PCT Filed: Sep. 1, 2016
(86) PCT No.: PCT/US16/49869

§ 371 Date: Mar. 1, 2018

Related U.S. Application Data
(60) Provisional application No. 62/214,695, filed on Sep. 4, 2015.

(51) Int. Cl. C12Q 1/18; C12Q 1/37; A61P 31/04; C12N 9/52; A61K 31/433; A61K 31/58; A61K 31/4192

(52) U.S. Cl. CPC C12Q 1/18; C12Q 1/37; A61P 31/04; A61K 31/4192; C12N 9/52; A61K 31/433; A61K 31/58; C12Y 304/23036

(57) ABSTRACT

This invention provides purified and recombinantly-produced bacterial lipoprotein signal peptidase (Lsp) enzymes and in vitro assays for monitoring Lsp catalytic activities. Also provided in the invention are screening methods for identifying novel antibiotic agents and their therapeutic applications for treating bacterial infections. Further provided in the invention are specific Lsp inhibitory compounds which can be used as bactericidal agents in treating diseases caused by bacterial infections.

### CROSS-REFERENCE TO RELATED APPLICATIONS

The subject patent application claims the benefit of priority to U.S. Provisional Patent Application No. 62/214,695 (filed Sep. 4, 2015). The full disclosure of the priority application is incorporated herein by reference in its entirety and for all purposes.

### BACKGROUND OF THE INVENTION

All bacteria are dependent on lipoproteins for a diverse array of essential roles, including nutrient uptake, signal transduction, cell wall stability, adhesion, and virulence. A covalent lipid modification anchors all lipoproteins to the bacteria cell membrane and the process of lipid attachment is entirely contingent on the integral membrane lipoprotein signal peptidase (Lsp). Lsp represents a remarkable target for the development of broad and novel antimicrobial agents, as it is highly conserved throughout the bacterial kingdom (both Gram⁻ and ⁺ phyla) and no human homologues exist. The latter criterion is of critical importance to eliminating promiscuity and off-target effects of molecules when administered to the host. This type of molecular specificity is best exemplified by the β-lactam antibiotic penicillin, which targets the enzyme DD-transpeptidase, a unique functionality limited to the bacterial world. Despite the numerous attributes of Lsp that makes the protein an attractive target for drug discovery, no small molecule inhibitors have been reported.

There is a strong need in the art for additional and better drugs for treating bacterial infections. The present invention is directed to this and other unfulfilled needs in the art.

### SUMMARY OF THE INVENTION

In one aspect, the present invention provides methods for assay systems for measuring catalytic activity of a lipoprotein signal peptidase (Lsp). The systems contain (a) a recombinantly-expressed, soluble and purified Lsp enzyme and (b) an Lsp substrate. In some of the systems, the Lsp is a bacterial Lsp such as E. coli Lsp. In some embodiments, the Lsp is expressed as a His-tagged fusion protein. In some embodiments, the Lsp is solubilized with a detergent, e.g., n-Dodecyl β-D-maltoside (DDM). In some embodiments, the substrate is a peptide, a peptide mimetic, or a protein that contains a lipid-modified cysteine residue. In some embodiment, the substrate is labeled with a fluorescence resonance energy transfer (FRET) donor-acceptor pair.

In another aspect, the invention provides methods for identifying agents that inhibit a lipoprotein signal peptidase (Lsp). The methods entail (a) contacting a recombinantly-produced and purified Lsp with an Lsp substrate in the presence of test compounds, and (b) detecting inhibition by one or more test compounds of Lsp cleavage of the substrate. In some embodiments, the Lsp is a bacterial Lsp such as E. coli Lsp. In some embodiments, the Lsp is expressed as a His-tagged fusion protein. In some embodiments, the Lsp is solubilized with a detergent, e.g., n-Dodecyl β-D-maltoside (DDM). In some embodiments, the substrate is a peptide, a peptide mimetic, or a protein that contains a lipid-modified cysteine residue. In some embodiment, the substrate is labeled with a fluorescence resonance energy transfer (FRET) donor-acceptor pair. In some embodiment, the Lsp catalytic activity is detected via fluorescence resonance energy transfer. In some embodiments, the screening is performed in a high throughput format. In some embodiments, test compounds are small organic compounds. Some methods of the invention additionally involve examining the identified agents for bactericidal activity.

In another aspect, the invention provides methods for inhibiting Lsp catalytic activity in a microbial cell (e.g., a bacterium). The methods entail contacting the microbial cell with an Lsp inhibitor compound under conditions to allow the compound to inhibit Lsp that is present in the cell, wherein the Lsp inhibitor compound is Compound BBS-8, Compound BBS-20, or any of the compounds shown in FIG. 12 such as Compound 01000270728-1, or a functional variant thereof. In some embodiments, the microbial cell in present inside a subject. In some of these embodiments, the subject is afflicted with an infection by the microbial cell.

In a related aspect, the invention provides methods for inhibiting a microbial (e.g., a bacterium) growth and for treating a microbial infection (e.g., bacterial infection) in a subject. These methods involve administering to the subject afflicted with a microbial infection a pharmaceutical composition comprising a therapeutically effective amount of an Lsp inhibitor compound. In these methods, the Lsp inhibitor compound is Compound BBS-8, Compound BBS-20, or any of the compounds shown in FIG. 12 such as Compound 01000270728-1, or a functional variant thereof. In some preferred embodiments, the subject is a human.

In another aspect, the invention provides methods for generating an active detergent-solubilized transmembrane enzyme capable of measuring catalytic activity in an assay system. The methods entail (a) constructing an expression vector capable of expressing the active transmembrane enzyme; (b) expressing the active transmembrane enzyme from the expression; and (c) solubilizing and purifying the active transmembrane enzyme in a detergent based system. In some of these methods, the employed transmembrane enzyme is Lsp. In some related embodiments, the invention provides uses of the transmembrane enzymes produced according to these methods in an assay to measure catalytic activity of the enzymes. Some of the uses relate to high throughput screen to identify specific inhibitors of a bacterial transmembrane enzyme, e.g., Lsp.

In another aspect, the invention provides methods for identifying an Lsp-inhibitory compound with improved properties. The methods entail (a) synthesizing one or more structural analogs of a lead Lsp inhibitor compound; (b) performing a functional assay on the analogs to identify an analog that has an improved biological or pharmaceutical property relative to that of the lead compound; thereby identifying an Lsp-inhibitory compound with improved properties. In some embodiments, the lead Lsp inhibitor compound is Compound BBS-8, Compound BBS-20, or any of the compounds shown in FIG. 12 such as Compound 01000270728-1, or a functional variant thereof. In some of these methods, the improved biological or pharmaceutical property is an enhanced inhibition of Lsp catalytic activity. In some methods, the functional assay utilizes a purified and detergent-solubilized Lsp enzyme.

A further understanding of the nature and advantages of the present invention may be realized by reference to the remaining portions of the specification and claims.

### BRIEF DESCRIPTION OF THE DRAWINGS

FIG. 1 is the schematic of an Lsp FRET peptide substrate.

FIG. 2 shows the scatterplot for a 10-plate Maybridge HitFinder™ assay.

FIG. 3 shows the scatterplot for a 40-plate Maybridge HitFinder™ assay.

FIG. 4 shows hit compounds from the Maybridge HitFinder™ screen and % inhibition of Lsp activity.

FIG. 5 shows inhibition by Sharpless compound library measured in triplicate.

FIG. 6 shows structures of hit compounds BBS-8 and -20.

FIG. 7 shows dose-dependent inhibition of Lsp by BBS-20.

FIG. 8 shows that BBS-20 inhibits Lsp by a non-competitive mechanism.

FIG. 9 shows some modifications to Compound BBS-20 for generating functional variants.

FIG. 10 is the schematic of the concentrations and volumes used in the ultra-high-throughput screen searching for Lsp inhibitors.

FIG. 11 shows data pertaining to the LSP primary and counterscreen titration assay results. Panels A, B and C of the figure shows the overall statistic summary of LSP Primary and Counterscreen Titration Assay and CRC of control compound in both assays. Panel D is cluster ranking of 344 compounds tested in the titration assays, which was plotted using Max % Response vs. Cluster ID. Note that there are 55 clusters among the 344 compounds, and the top 17 hits are shown in red dots. Structures of representative top hits are shown close to the red dots of each cluster.

FIG. 12 shows examples of compounds identified in the Counterscreen Titration Assay to inhibit Lsp in a dose-dependent manner.

FIG. 13 shows synthesis and in vitro validation of Compound SR-01000270728-1 with a dose-dependent assay.

### DETAILED DESCRIPTIONS

### I. Overview

Despite the long felt need in the art, there are currently no Lsp-specific inhibitors. This may be attributed to the tremendous difficulty associated with the purification and assay development of an active transmembrane enzyme. The invention is predicated in part on the development by the present inventors of the first in vitro high-throughput screen (HTS) for an integral membrane protease. As detailed herein, the inventors successfully expressed, purified and solubilized E. coli lipoprotein signal peptidase (Lsp). The inventors additionally developed an in vitro assay to monitor Lsp cleavage activity based on fluorescence resonance energy transfer (FRET). The assay utilized a lipoprotein mimetic peptide substrate which is labeled with a fluorophore and fluorescence quencher. The inventors optimized the HTS by selecting enzyme, substrate, and library compound concentrations capable of identifying all possible inhibition modalities (i.e., competitive and non-competitive). Further, the inventors performed a provisional pilot screen of 15,000 compounds from the Maybridge HitFinder collection. An additional screen of an internal library (“Sharpless compound library”) resulted in identification of lead compounds, which were validated as Lsp inhibitors in in vitro functional assays. These results demonstrated that Lsp is a viable target for drug discovery, and that the assay is reproducible and robust, as highlighted by an ample signal-to-background and Z-prime well above the statistically significant value of 0.5. As further validation of utilities of the purified Lsp enzyme and the in vitro screening system described herein, the inventors further performed ultra-high throughput screening and validation assays for Lsp inhibitors. This screen employed a 1,536-well format and screened a library of 646,275 candidate compounds. As detailed herein, a total of 2,271 active compounds were obtained from the primary assay. With secondary assays, about 344 compounds were found to demonstrate selective activity. Properties of exemplary compounds were confirmed with further synthesis and validation assays.

In accordance with these studies, the present invention provides novel assay systems for monitoring and quantifying Lsp enzymatic activities. Also provided in the invention are methods for identifying novel agents for inhibiting Lsp enzymatic activities and inhibiting bacterial growth. Such agents provide novel antibiotics that can be broadly employed to treat bacterial infections. The invention additionally provides specific Lsp-inhibitory compounds which can be used as bactericidal agents against Gram⁻ and Gram⁺ organisms. Further provided in the invention are methods of using the identified Lsp-inhibitory molecules as lead compounds to identify additional antibiotic agents with improved biological and/or pharmaceutical properties. The following sections provide further guidance for making and using the compositions of the invention, and for carrying out the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

The term “contacting” has its normal meaning and refers to combining two or more agents (e.g., polypeptides or small molecule compounds) or combining agents and cells (e.g., a small molecule and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

EDANS, 5-[(2-aminoethyl)amino]naphthalene-1-sulfonic acid, is one of the most popular donors for developing FRET-based nucleic acid probes and protease substrates. EDANS is often paired with DABCYL or DABSYL in FRET-based probes. Its fluorescence is environment-sensitive. Dabsyl (dimethylaminoazobenzenesulfonic acid) absorbs in the green spectrum and is often used with fluorescein. It is a dark quencher which is a substance that absorbs excitation energy from a fluorophore and dissipates the energy as heat. While a typical (fluorescent) quencher re-emits much of this energy as light. Dark quenchers are used in molecular biology in conjunction with fluorophores. When the two are close together, such as in a molecule or protein, the fluorophore's emission is suppressed. This effect can be used to study molecular geometry and motion.

Globomycin is a cyclic peptide antibiotic that inhibits the growth of Gram-negative bacteria, such as E. coli. See, e.g., Inukai et al., J. Antibiot. 31: 410-420, 1978. Globomycin inhibits Lsp and causes the accumulation of diacylglyceryl prolipoproteins in the inner membrane. Thus, Gram-negative organisms are sensitive to globomycin due to inhibition of murein prolipoprotein processing to lipoprotein.

As used herein, IC₅₀ refers to the concentration of a compound at which a half-maximal inhibition of an enzymatic activity is reached.

The terms “identical” or “sequence identity” in the context of two nucleic acid sequences or amino acid sequences refers to the residues in the two sequences which are the same when aligned for maximum correspondence over a specified comparison window. Methods of alignment of sequences for comparison are well known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). Alignment is also often performed by inspection and manual alignment.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (e.g., BLAST) using standard parameters. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

Bacterial lipoproteins are characterized by their fatty-acylated amino termini via which they are anchored into lipid membranes. They have a wide variety of biological functions in bacteria, such as maintenance of cell envelope architecture (Lpp and Pal), insertion and stabilization of outer membrane proteins (BamB), uptake of nutrients and metals (OppA and SitC), protein folding (PrsA), bacteriocin release (BRP), and adhesion and invasion (OspC and Lmb). Lipoproteins, which constitute 2 to 3% of bacterial proteomes, are synthesized in the cytoplasm as prolipoproteins and contain a conserved lipoprotein signature motif called lipobox that allows recognition by the lipoprotein modification machinery. The invariant cysteine +1 becomes the first amino acid of the mature protein after modification; residues −4 to −1 are cleaved off as part of the signal peptide.

Lipoproteins are inserted into the membrane and modified on the membrane by the sequential action of three membrane-bound enzymes. The first step is catalyzed by lipoprotein diacylglyceryl transferase (Lgt), which catalyzes the formation of a thioether bond formation between a conserved cysteine residue and a diacylglycerol (DAG) moiety derived from membrane phosphatidylglycerol. This results in the formation of a thioether-linked diacylglyceryl-prolipoprotein and glycerolphosphate as a by-product. Following lipid attachment, lipoprotein signal peptidase (Lsp) removes a signal peptide by cleaving diacylglyceryl-prolipoprotein at the amino-terminal end of the diacylated cysteine residue, leaving the DAG-modified cysteine as the new N-terminus of the newly formed apolipoprotein. A third enzyme, lipoprotein N-acyltransferase (Lnt), transfers an additional acyl group from a membrane phospholipid to the newly-generated α-amino group of the lipid-modified cysteine, generating a fully mature triacylated lipoprotein. The enzymes Lgt and Lsp are conserved in all classes of bacteria, whereas Lnt is only present in Gram negative bacteria and some Gram positive species. All three enzymes have been shown to play essential roles in the survival of E. coli and other Gram-negative bacteria. By contrast, in Gram-positive bacteria, Lgt and Lsp appear to be essential in at least some of the tested Actinobacteria [high-guanine+cytosine (GC)-content species] but not in Firmicutes (low-GC-content species).

Lipoprotein signal peptidase (Lsp), also termed “prolipoprotein signal peptidase”, “signal peptidase II”, “premurein-leader peptidase” and “leader peptidase II”, cleaves the signal peptide present in front of the lipidated cysteine residue of prolipoproteins. As exemplification, E. coli Lsp is an integral membrane protein with four transmembrane segments. Both its N-terminus and C-terminus face the cytoplasm. Two conserved aspartic acid residues (D102 and D129 in B. subtilis Lsp) in the type II signal peptidases of 19 bacterial species including E. coli are critical for the Lsp activity of both B. subtilis and S. coelicolor. These two aspartic acids might act as a catalytic dyad for a pepsin-type aspartic protease. E. coli Lsp strictly cleaves peptide bonds at the N-terminus of the lipid-modified cysteine residue, whereas Lsps from some Gram-positive bacteria may have a lower specificity or a different recognition mode for the substrate. The enzymatic activity of Lsp can be inhibited noncompetitively by the cyclic depsipeptide antibiotic globomycin.

The term “modulation” or “modulating” refers to the activity of a compound or other agent in evoking a change in a biological activity of, or a functional response mediated by, another molecule (e.g., an Lsp enzyme). The term “modulate” refers to a change in the biological or cellular activities (e.g., enzymatic or signaling activities) of the target molecule. Modulation can be up-regulation (i.e., activation or stimulation) or down-regulation (i.e. inhibition or suppression). For example, modulation may cause a change in reduced catalytic activity of a target enzyme (e.g., Lsp), or any other biological activities or functions of, or cellular or immunological activities mediated by, the target molecule (e.g., an enzyme's binding to substrate). The mode of action may be direct, e.g., through binding to the target molecule. The change can also be indirect, e.g., through binding to and/or modifying (e.g., enzymatically) another molecule which otherwise modulates the target molecule.

“Purified” means that a material (e.g., an Lsp protein or fragment thereof) has been removed from the environment in which it was made. A material may be partially or substantially purified and need not be completely (100%) pure. For example, an Lsp protein of the invention may be purified after it has been recombinantly synthesized by removing some or all of the unreacted chemicals, side products, cellular debris and other components. As used herein, “substantially purified” or “substantially pure” means that the material is at least 75%, 80%, 85%, 90%, 95% or 99% free of other substance or components.

The term “subject” refers to mammals, particularly humans. It encompasses other non-human animals such as cows, horses, sheep, pigs, cats, dogs, mice, rats, rabbits, guinea pigs, monkeys.

A “variant” of a molecule refers to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical. As used herein, a functional variant or functional derivative refers to a variant of a reference molecule (e.g., an Lsp enzyme) that shares a similar biological function (e.g., catalytic function) as that of the reference molecule.

### III. Recombinantly-Produced, Solubilized and Purified Lsp Proteins

The invention provides purified and solubilized Lsp proteins that are recombinantly-produced as described herein. As exemplified herein, some of the purified Lsp proteins are detergent-solubilized. Also provided in the invention are uses of these functional Lsp enzymes in assay systems for monitoring Lsp catalytic activity. Lsps are expressed in various bacterial, mycoplasma and archaea species. Lsps from any of these species can be expressed and purified in accordance with the methods described herein. In some preferred embodiments, Lsps used in the practice of the invention are from bacteria, including both G⁺ and G⁻ bacterial species. Lsps are well conserved in many bacterial species. These include, e.g., E. coli and species of Enterobacter, Pseudomonas, Mycobacterium, Listeria, Streptococcus, and Staphylococcus. Sequences and structures of Lsp from a number of bacterial species are all known and characterized in the art. See, e.g., Innis et al., Proc. Natl. Acad. Sci. USA 81: 3708-3712, 1984; Isaki et al., J. Bacteriol. 172: 469-472, 1990; Reglier-Poupet et al., J. Bacteriol. 158:632-635, 2003; Sander et al., Mol. Microbiol. 52:1543-1552, 2004; Witke et al., FEMS Microbiol. Lett. 126:233-239, 1995; De Greeff et al., Microbiol. 149:1399-407, 2003; Zhao et al., FEBS Lett. 173: 80-84, 1992. Additional description of the structural information of various Lsp enzymes is also available in the art. These include sequences of Lsp from R. typhi (Rt) (GenBank accession no. NC_006142), R. prowazekii (Rp) (GenBank accession no. AJ235271), R. bellii (Rb) (GenBank accession no. NZ AARC01000001), R. canadensis (Rcan) (GenBank accession no. NZ_AAFF01000001), R. akari (Ra) (GenBank accession no. NZ_AAFE01000001), R. conorii (Rc) (GenBank accession no. NC_003103), R. sibirica (Rs) (GenBank accession no. AABW01000001), R. rickettsii (Rr) (GenBank accession no. NZ AADJ01000001), R. felis (Rf) (GenBank accession no. NC_007109), and E. coli (Ec) (GenBank accession no. X00776). Any of these Lsp sequences or substantially identical sequences thereof can be employed in producing recombinant Lsp or variants in the present invention.

The general techniques of molecular biology and biochemistry well known in the art (e.g., PCR and affinity chromatography) can be utilized in the cloning, expression and purification of the Lsp proteins of the invention. Such routinely practiced methods and techniques are described, e.g., in Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003). However, some specific protocols for expressing and purifying functional soluble Lsp enzymes are developed by the present inventor and described in detail in the Examples herein. Using E. coli Lsp as an example, the full-length protein can be generated by first cloning its coding sequence via colony PCR. The E. coli Lsp coding sequence is shown in SEQ ID NO:3 herein. To facilitate subsequent purification, Lsp can be overexpressed as a fusion protein with an appropriate tag. As discovered by the present inventors for E. coli Lsp, an N-terminal His-tag greatly facilitated the Lsp expression and purification. This can be readily achieved with a suitable expression vector and host cell line, e.g., pET19b vector and the E. coli BL21(DE3)pLysS (Agilent) cells. Other than N-terminal His-tag exemplified herein, the recombinant expression and purification strategy of the invention can also utilize a C-terminal His-tag, which can be similarly cleaved by proteolytic cleavage. After overexpression, the cells are lysed and an appropriate agent can be used to solubilize the membrane proteins. It was found that some specific detergents, e.g., n-Dodecyl β-D-maltoside (DDM), allow optimal solubilization of the protein for ensuring purification and maintenance of its structural integrity. As exemplified herein, the protein expression and purification scheme described herein, including the use of the N-terminal His-tag and the solubilization detergent, enables efficient purification of the protein by affinity column and gel filtration chromatography. This also led to a purified and intact soluble membrane protein Lsp which retains its enzymatic activities.

As Lsp is highly conserved among both Gram⁺ and Gram⁻ bacterial species, the same recombinant expression and purification scheme can be readily applied for obtaining soluble and functional recombinant Lsp proteins from a variety of other bacterial species. Thus, in addition to E. coli Lsp enzyme, Lsp proteins from any other species can be similarly cloned into a pET vector (e.g., pET19b) and purified as described herein for E. coli Lsp. Indeed, the present inventors also cloned and expressed the Lsp enzyme from a number of other species. For example, as demonstrated herein, the same protocols developed for E. coli Lsp were successfully employed to clone, express, and purified Lsp from Streptococcus pyogenes and Thermotoga maritima. Specifically, the Lsp protein of Streptococcus pyogenes strain M1GAS was expressed and purified by cloning the coding sequence (SEQ ID NO:4) into vector pET23b via NdeI and XhoI cloning sites as a C-terminal His₆-tagged fusion. Similarly, Lsp from Thermotoga maritima strain MSB8 was expressed and purified by cloning the coding sequence (SEQ ID NO:5) into vector pET19b via NdeI and BamHI cloning sites as a N-terminal His₆-tagged protein.

### IV. Assay Systems and Screening Methods for Solubilized Membrane Proteins

The invention provides assay systems that utilize an active detergent-solubilized enzyme as exemplified herein for Lsp. The assay systems and related screening methods can be employed for measuring catalytic activity of many other membrane proteins and for screening modulators thereof. In addition to the enzymes involved in bacterial lipoprotein biogenesis described herein (including Lsp), many other membrane enzymes known in the art may also be suitable for the assay systems and screening methods of the invention. Examples include hydrolases, phospholipases (e.g., Phospholipase A and C), cholesterol oxidases, lipoxygenases, carotenoid oxygenase, ferrochelatase, glycolate oxidase, glycosyltransferases, and etc. Utilizing appropriate substrates well known in the art for these enzymes, each of these enzymes can be examined in assay systems and screening methods similar to that for Lsp exemplified herein.

To obtain the assay systems or to perform the screening methods, typically an expression construct is first generated which is capable of expressing the active transmembrane enzyme as exemplified herein for Lsp. This is followed by expressing the active transmembrane enzyme from the expression construct. The expressed transmembrane enzyme is then solubilized and purified in a detergent based system, which allows the formation of an active transmembrane detergent-solubilized enzyme. The functional detergent-solubilized enzyme can then be employed for measuring the specific catalytic activity in the assay systems or screening methods of the invention. As demonstrated herein for Lsp, the assays systems can be used for measuring the catalytic activity of the detergent-solubilized enzyme. In some embodiments, the detergent-solubilized enzyme is used in a high throughput screen to identify specific inhibitors of the enzyme. As described herein, such inhibitors can be identified from various candidate compounds, e.g., small molecules, peptides, polypeptides or chimeric versions thereof. Some embodiments of the invention use a detergent-solubilized Lsp as exemplified herein to screen for specific inhibitors of Lsp that have antimicrobial activity.

As exemplification, the invention provides assay systems that employ a detergent-solubilized active Lsp enzyme described herein for monitoring Lsp catalytic activities. In addition to the recombinantly-produced and purified Lsp proteins, the assay systems typically also contain an Lsp substrate and optionally a means that can detect a catalytic event of the enzyme on the substrate. The substrate can be any peptide, polypeptide or peptide mimetic that can be recognized and specifically cleaved by the enzymatic function of Lsp. The substrate typically contains an Lsp cleavage site, i.e., a lipidated cysteine residue. In some embodiments, the substrate is conjugated to a label moiety that allows for detection of a cleavage event. The label moiety can be a molecule with fluorescent properties which alter upon cleavage from the substrate, or a matched donor-acceptor pair of fluorescence resonance energy transfer (FRET) compounds. In some embodiments, a fluorescence donor moiety and a fluorescence acceptor moiety pair are attached to the substrate peptide on opposite sides of the Lsp cleavage site, such that monitoring the cleavage of the substrates is performed by detecting a fluorescence resonance energy transfer. Monitoring can include detecting a shift in the excitation and/or emission maxima of the fluorescence acceptor moiety, which shift results from release of the fluorescence acceptor moiety from the substrate by the Lsp peptidase activity.

In some embodiments, Lsp catalytic activity is detected and quantified via a fluorescence resonance energy transfer (FRET) assay by monitoring fluorescence signal resulting from cleavage of a labeled substrate peptide. FRET is a non-radiative process that energy from a donor is transferred to an acceptor when they have overlapping emission/absorption spectra with a suitable orientation and distance (e.g., in the range of 10-100 Å). Any fluorescence resonance transfer energy pair (fluorophore and fluorescence quencher) known in the art can be used to label the Lsp peptide substrate. In some preferred embodiments, the assay system can utilize a substrate peptide mimetic that is labeled with the FRET donor-acceptor pair of EDANS and Dabsyl as exemplified herein. In addition to this exemplified labels, other FRET donor-acceptor pairs known in the art may also be used in the practice of the invention. For example, green fluorescent protein (GFP) is a spontaneously fluorescent protein which has been commonly adopted as an excellent reporter module of the fusion proteins. The most important feature of GFP is that variants of GFP have showed distinguished spectral properties which can be used as donors and acceptors of FRET. The original pair of fluorescent proteins was a blue fluorescent protein (BFP) donor and a GFP acceptor with relatively low quantum yield, easy bleaching, and high autofluorescence background (Heim, Methods Enzymol., 302: 408-423, 1999). As improvements, a pair of GFP mutants with longer wavelengths, namely cyan fluorescent protein (CFP) and yellow fluorescent protein (YFP), has been shown to have better FRET efficiency (see, e.g., Miyawaki et al., Nature 388: 882-887, 1997). In addition, red fluorescent proteins from corals have also been cloned and paired with YFP to create red-shifted excitation and emission peaks (see, e.g., Mizuno et al., Biochemistry, 40: 2502-2510, 2001). Further examples of FRET donor-acceptor pairs that may be used in the practice of the invention include amino benzoic acid and nitro-tyrosine; 7-methoxy-3-carbamoyl-4-methylcoumarin and dinitrophenol; or 7-dimethylamino-3-carbamoyl-4-methylcoumarin and dabsyl.

In a related aspect, the invention provides screening methods for identifying agents that are capable of inhibiting Lsp enzymatic activity. According to the present invention, novel inhibitors of bacterial Lsp are typically identified in vitro in a high-throughput screen (HTS) format. The screening methods utilize the assay systems described above, which contain a purified Lsp enzyme such as E. coli Lsp (or a functional variant or fragment) and a lipidated protein or peptide substrate, and monitor Lsp catalytic activity in the presence of test agents or candidate compounds. To allow detection of enzymatic activity, the substrate can be labeled with a fluorophore and fluorescence quencher. Lsp cleavage activity is quantified based on fluorescence resonance energy transfer (FRET). As exemplified herein, the Lsp enzyme is detergent-solubilized to facilitate the catalytic reaction on the substrate protein or peptide mimetic. In additional to the specific protocols for carrying out the screening methods detailed below, various general biochemical and molecular biology techniques or assays well known in the art can be employed in the screening methods of the invention. Such techniques are described in, e.g., Handbook of Drug Screening, Seethala et al. (eds.), Marcel Dekker (1^{st} ed., 2001); High Throughput Screening: Methods and Protocols (Methods in Molecular Biology, 190), Janzen (ed.), Humana Press (1^{st} ed., 2002); Current Protocols in Immunology, Coligan et al. (Ed.), John Wiley & Sons Inc. (2002); Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003).

As exemplified in Examples 3 and 4 herein, the high-throughput screening format developed by the inventors allows identification of Lsp modulating agents by performing the Lsp catalytic assay simultaneously in the presence of each member of a library of test agents. By detecting a downregulated Lsp catalytic activity in the presence of a test agent, a potential or candidate Lsp inhibitor can then be obtained from the test agents. To be considered a candidate Lsp inhibitor, the downregulated Lsp activity typically should represent a significant departure from a baseline Lsp activity that is obtained from the assay performed in the absence of any of the test compounds. A departure from a base line level or activity is considered significant if the determined level falls outside the range typically observed with control compounds known to have no effect on Lsp enzymatic function, due to inherent variation between compounds and experimental error. For example, in some methods, a departure can be considered significant if a determined level does not fall within the mean plus one standard deviation of levels in control compounds. Typically, a significant departure occurs if the difference between the measured level and baseline levels is at least 20%, 30%, or 40%. Preferably, the difference is by at least 50% or 60%. More preferably, the difference is more than at least 70% or 80%. Most preferably, the difference is by at least 90%. The extent of departure between a determined value and a standard or baseline value in control compounds also provides an indicator of the likely reliability or inhibitory function of the identified hit compounds. In some embodiments, the screening methods can additionally employ a known Lsp inhibitor (e.g., globomycin) in the screening assay as a positive control to evaluate likely activity of the identified hits.

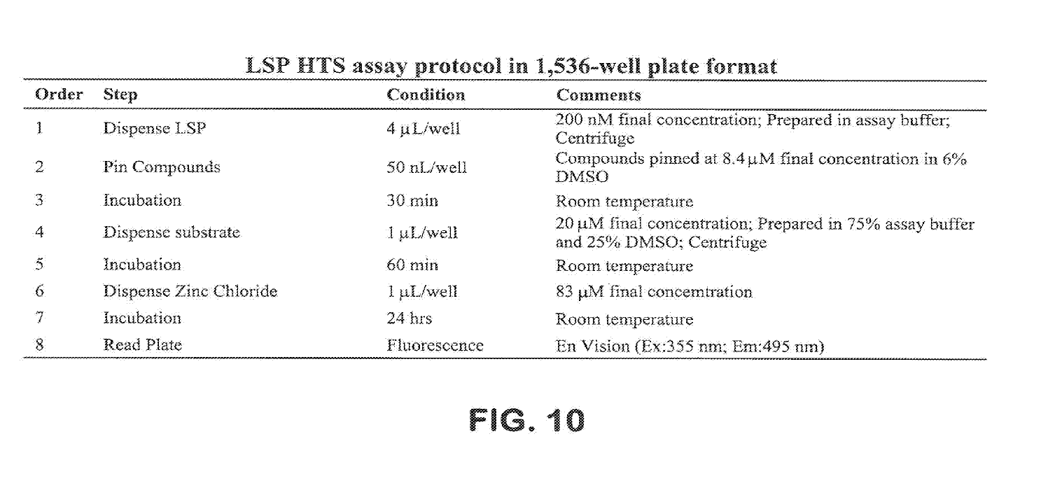

Once hit compounds are identified from the initial screen, they can typically be subject to further screening or functional validation. Thus, in some embodiments, the hit compounds can be further tested in vitro for their ability to inhibit Lsp enzymatic activity, as exemplified herein for Compounds BBS-8 and BBS-20 (FIG. 6) and Compound SR-01000270728-1 (FIGS. 14 and 15). In some embodiments, hit compounds that pass such in vitro validation can be examined for bactericidal activities. This can be performed with a high-throughput assay that is sensitive enough to detect cells that have been killed due to contact with the compounds. In some embodiments, the candidate Lsp inhibitors can be examined for bactericidal activity via the well-known kill curve assays using a panel of both G⁺ and G⁻ bacteria species. See, e.g., Sanfilippo et al., Chemother. 18:297-303, 1973. In some embodiments, bacterial killing can be detected by examining one or more viability indicators via a suitable means. The viability indicators can be any signal that can be used to distinguish live cells from dead cells, or to distinguish cells that are damaged but alive from cells that are undamaged and alive. In some embodiments, the viability indicators are examined by monitoring an optical signal which correlates with the cell viability indicators. For example, fluorescence-based assays can be used for evaluating bacterial viability. Some of these assays use nucleic acid stains to differentiate between live and dead cells. Many of the assays and the employed stains can be obtained commercially, e.g., the LIVE/DEAD BacLight Bacterial Viability Kit from Molecular Probes (Eugene, Oreg.) and BacTiter-Glo™ assay from Promega. Additional assays for examining bactericidal activities of the Lsp-inhibitory compounds of the invention are described in the art, e.g., Roth et al., Appl. Environ. Microbiol. 63:2421-2431, 1997.

In some embodiments, the identified candidate inhibitors can be further screened for ability to inhibit other enzymes catalyzing bacterial lipoprotein biogenesis. For example, the identified candidate Lsp inhibitors can be tested for ability to inhibit the enzymatic function of lipoprotein diacylglyceryl transferase (Lgt). Alternatively, test compounds may be first screened for Lgt-inhibitory agents prior to being examined for Lsp-inhibitory function. In some embodiments, test compounds may be screened simultaneously screened for activities in inhibiting both Lgt and Lsp. Candidate compounds with such dual inhibitory activities can be further analyzed for bactericidal function. In some other embodiments, candidate Lsp inhibiting compounds identified from the initial screening can be modified, e.g., by rational design, to generate analogs or derivative compounds that possessing improved or desired physiochemical or pharmaceutical properties. Such analog or derivative compounds may then be subject to subsequent screening or further functional examination described herein.

In addition to an intact Lsp molecule or nucleic acid encoding the intact Lsp molecule, an Lsp functional fragment (e.g., fragments harboring the substrate binding domain and the catalytic domain), analog, derivative, or a variant protein with substantially identical sequence can also be used in the screening methods of the invention. The Lsp fragments that can be employed in these assays usually retain one or more of the biological activities of the Lsp molecule (e.g., its peptidase activity). As noted above, Lsps from the different species have already been sequenced and well characterized, including delineation of the active site of the enzyme. See, e.g., Jjalsma et al., J. Biol. Chem. 274:28191-28197, 1999. Therefore, their fragments, analogs, derivatives, or fusion proteins suitable for the invention can be easily obtained using methods well known in the art. For example, a functional derivative of an Lsp can be prepared from the recombinantly-produced Lsp protein described herein via proteolytic cleavage followed by conventional purification procedures known to those skilled in the art. Alternatively, the functional derivative can be produced by recombinant DNA technology by expressing only fragments of an Lsp that retain its substrate binding and enzymatic activity.

### V. Test Compounds

Test compounds or candidate agents that can be screened with methods of the present invention include small molecule organic compounds, polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules while others are natural molecules.

Test agents can be obtained from a wide variety of sources including libraries of synthetic or natural compounds. Combinatorial libraries can be produced for many types of compound that can be synthesized in a step-by-step fashion. Many libraries of small organic molecules are publicly or commercially available or otherwise accessible for drug screening. Examples include the Maybridge HitFinder library (Thermo Fisher), the Molecular Libraries Small Molecule Repository (NIH), and several small molecule compound libraries from Selleckchem (Boston, Mass.). Such libraries can also be synthesized as described in the art, e.g., Carell et al., Chem. & Biol. 2: 171-183, 1995. Large combinatorial libraries of small molecule compounds can also be constructed by the “DNA-encoded chemical libraries” (DEL) or “encoded synthetic libraries” (ESL) method. This is a technology for the synthesis and screening of collections of small molecule compounds of unprecedented size. DEL is used in medicinal chemistry to bridge the fields of combinatorial chemistry and molecular biology. Detailed procedures for constructing DEL libraries are described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of small molecules, peptides or other compounds can be fully randomized, with no preferred groups in the compounds or sequence preferences or constants at any position. Alternatively, the library can be biased, i.e., with some groups in the organic compounds or positions within the peptide sequences being held constant. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins. The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules, e.g., molecules with a molecular weight of not more than about 500 or 1,000. Preferably, high throughput assays are adapted and used to screen for such small molecules. In some methods, combinatorial libraries of small molecule test agents as described above can be readily employed to screen for small molecule modulators of Lsps via the assay systems described herein. Some general guidance for screening combinatorial libraries of small molecule compounds is also provided in the art. See, e.g., Schultz et al., Bioorg. Med. Chem. Lett 8:2409-2414, 1998; Weller et al., Mol. Divers. 3:61-70, 1997; Fernandes et al., Curr. Opin. Chem. Biol. 2:597-603, 1998; and Sittampalam et al., Curr. Opin. Chem. Biol. 1:384-91, 1997. An exemplary library of small molecule compounds suitable for the high throughput screening methods of the invention is described in Example 4 below.

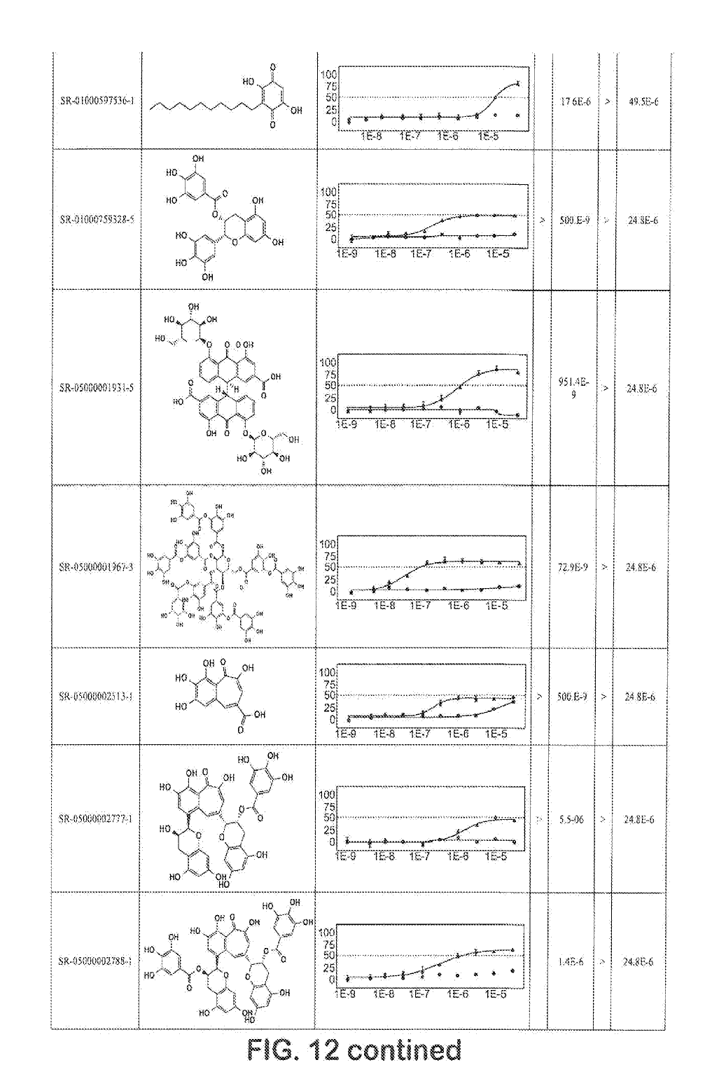

In some embodiments, the test agents employed in the screening methods of the invention are analogs or derivative compounds that are generated from a known compound. For example, the test agents can be derivatives or analogs of globomycin. Globomycin is an Lsp inhibitor. Globomycin is a peptide antibiotic that is made by several Streptomyces species and inhibits Gram-negative bacteria through the inhibition of Lsp. Globomycin derivatives have also been shown to have potent activity against Gram-positive bacteria. In some other embodiments, the test agents can be analogs derived from the specific Lsp-inhibiting compounds identified herein, e.g., Compound BBS-8, Compound BBS-20, or any of the compounds shown in FIG. 12 such as Compound 01000270728-1. Analogs or derivative compounds based on these base compounds can be prepared in accordance with the disclosure provided below. The analog or derivative compounds of the known compound are typically screened to identify agents with improved biological or pharmaceutical properties relative to the known compound.

Libraries of test agents to be screened with the claimed methods can also be generated based on structural studies of the Lsp enzyme, their fragments or analogs. Such structural studies allow the identification of test agents that are more likely to bind to the Lsp polypeptides. The three-dimensional structure of an Lsp polypeptide can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of an Lsp polypeptide structure provides another means for designing test agents for screening Lsp inhibitors. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”.

In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wtithrich (Wiley-Interscience, New York 1986).

### VI. Novel Lsp Inhibitors and Analogs Thereof with Improved Properties

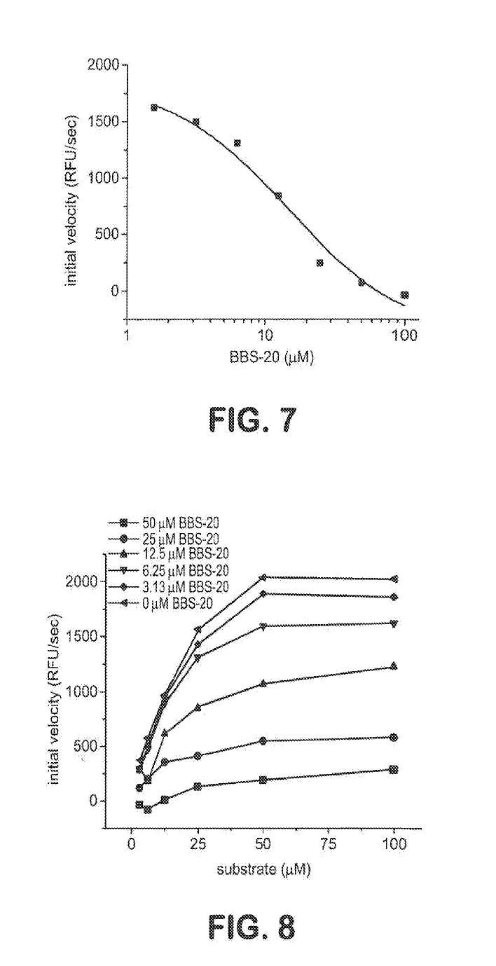

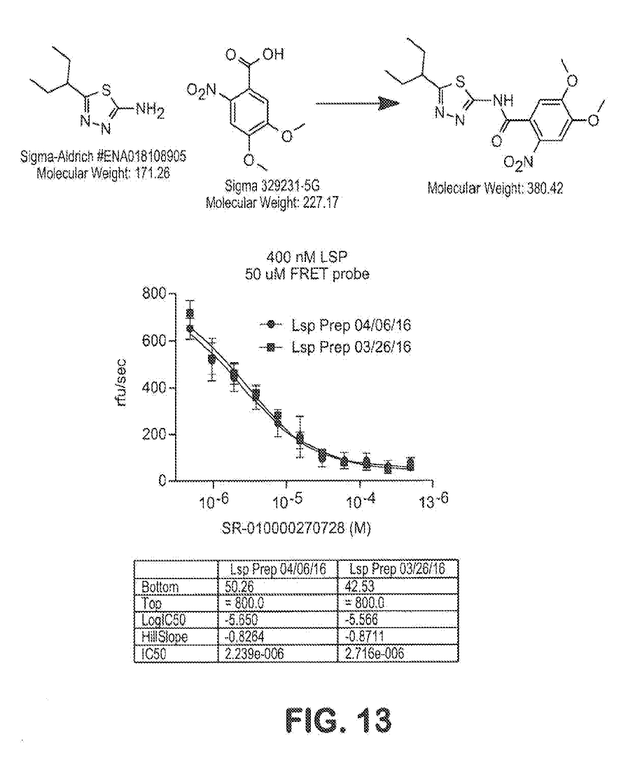

The in vitro assay systems of the invention for monitoring and quantifying Lsp catalytic activity enabled the inventors to identify novel Lsp inhibitory compounds. Two examples of such novel Lsp inhibitory compounds identified from a pilot screen are 3-(4-((8R,9S,13S,14S)-3-((fluorosulfonyl)oxy)-17-hydroxy-13-methyl-7,8,9,11,12,13,14,15,16,17-decahydro-6H-cyclopenta[a]phenanthren-17-yl)-1H-1,2,3-triazol-1-yl)-N,N,N-trimethylpropan-1-aminium (aka “BBS-8” herein) and 24442-(diethylamino)ethyl)-1H-1,2,3-triazol-1-yl)-N-(((1R,4aS,10aR)-7-isopropyl-1,4a-dimethyl-1,2,3,4,4a,9,10,10a-octahydrophenanthren-1-yl)methyl)acetamide (aka “BB S-20” herein). As detailed in the Examples, these two hits identified from the screening were further validated in vitro by assessing their inhibitory effect on Lsp catalytic function using varying concentrations of the compounds. A large number of additional Lsp inhibitor compounds were obtained via a further ultra-high throughput screening format as detailed in Example 4 herein. Among active compounds identified from the screen, selective Lsp-inhibiting activities of some of the compounds were confirmed by secondary assays (FIG. 12). One of these additional Lsp inhibitors, Compound SR-010000270728-1, demonstrated excellent functional profiles and structural properties (FIG. 13). Lsp-inhibiting activities of some other Lsp inhibitors identified herein are shown in FIGS. 4 and 14. The specific Lsp inhibiting compounds of the invention, their analogs, and functional derivatives or variants can all be used as antimicrobial agents or therapeutic agents, as described herein.

With the discovery of the lead Lsp-inhibiting compounds and also availability of the in vitro Lsp assay systems described herein, the invention provides screening methods for identifying analogs or derivatives of a known Lsp-inhibiting compound with improved properties. An important step in the drug discovery process is the selection of a suitable lead chemical template upon which to base a chemistry analog program. The process of identifying a lead chemical template for a given molecular target typically involves screening a large number of compounds (often more than 100,000) in a functional assay, selecting a subset based on some arbitrary activity threshold for testing in a secondary assay to confirm activity, and then assessing the remaining active compounds for suitability of chemical elaboration.

The Lsp-inhibiting compounds described herein, e.g., compounds shown in FIGS. 4, 6 and 14, as well as other known Lsp inhibitors (e.g., globomycin), provide lead compounds to search for related compounds that have improved biological or pharmaceutical properties. For example, analogs or derivatives of these compounds can be screened for to identify compounds that have a higher affinity for Lsp, a better inhibitory profile, or an enhanced in vitro or in vivo stability. Compounds with such improved properties can be more suitable for various pharmaceutical applications. In other embodiments, such analogs or derivative compounds can be used as the test agents in the second or subsequent rounds of screening methods of the invention.

These methods typically involve synthesizing analogs, derivatives or variants of a known Lsp inhibitor (e.g., Compound SR-010000270728-1, Compound BBS-8 or BBS-20). Often, a library of structural analogs of the Lsp inhibitor is prepared for the screening. A functional assay is then performed to identify one or of the analogs or derivatives that have an improved biological property relative to that of the lead compound from which the analogs or variants are derived. In some embodiments, the analogs are screened for an enhanced ability to inhibit Lsp catalytic activity. In some embodiments, they can be assayed to identify compounds with better pharmaceutical properties, e.g., stability.

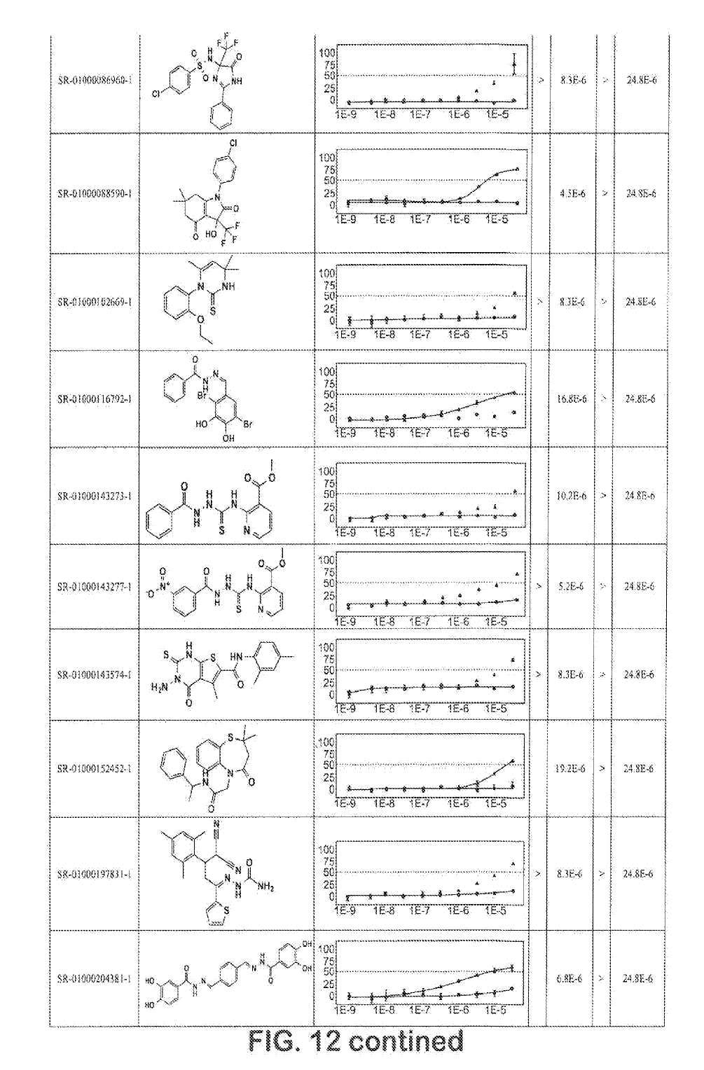

To synthesize analogs or derivatives based from the chemical backbones of the known or presently described Lsp inhibitors, only routinely practiced methods of organic chemistry that are well known to one of ordinary skill in the art are required. In some embodiments, analogs of a known compound can be generated by modifying the compounds in accordance with the common “click” chemistry as described in, e.g., Rostovtsev et al., Angew. Chem. Int. Ed. 41:2596-2599, 2002; and Himo et al., J. Am. Chem. Soc. 127: 210-216, 2005. As exemplification, some modifications that can be made to Compound BBS-20 to generate analogs or derivative compounds are shown in FIG. 9. In some embodiments, combinatorial libraries of chemical analogs of a known compound can be produced using methods described above. Exemplary methods for synthesizing analogs of various compounds are described in, e.g., by Overman, Organic Reactions, Volumes 1-62, Wiley-Interscience (2003); Broom et al., Fed Proc. 45: 2779-83, 1986; Ben-Menahem et al., Recent Prog. Horm. Res. 54:271-88, 1999; Schramm et al., Annu. Rev. Biochem. 67: 693-720, 1998; Bolin et al., Biopolymers 37: 57-66, 1995; Karten et al., Endocr. Rev. 7: 44-66, 1986; Ho et al., Tactics of Organic Synthesis, Wiley-Interscience; (1994); and Scheit et al., Nucleotide Analogs: Synthesis and Biological Function, John Wiley & Sons (1980).

In addition, any of the routinely practiced assays (e.g., binding assays) can be used to identify an improved property (e.g., enhanced binding affinity for Lsp or inhibiting profile) in analogs or derivatives of an Lsp inhibitor. Additional biochemical or pharmaceutical assays that can be employed are also well known and routinely practiced in the art. For example, improved stability of analog compounds can be assayed using methods such as those described in, e.g., Di et al., Comb. Chem. High Throughput Screen. 11:469-76, 2008; and Remington's Pharmaceutical Sciences, 18th ed., Mack Publishing Co. (1990).

### VII. Therapeutic Applications

The HTS assays of the invention for Lsp inhibitors enables identification of novel antibiotic agents with potent bactericidal activity against Gram⁺ and Gram⁻ organisms. Indeed, the naturally occurring cyclic peptide globomycin, which was shown to inhibit Lsp through a non-competitive mechanism just like Compound BBS-20 exemplified herein, is bactericidal. Thus, the specific Lsp-inhibiting compounds described herein (e.g., Compound BBS-8, Compound BBS-20, or any of the compounds shown in FIG. 12 such as Compound 01000270728-1), as well as their analogs or functional variants (e.g., compounds shown in FIG. 9), can be used in various therapeutic applications. In some embodiments, these compounds can be used to inhibit Lsp catalytic activity in microbial cells (e.g., bacteria) or to inhibit microbial growth. The microbial cells can be present either in vitro or in vivo (in a subject). In some embodiments, the invention provides methods for treating bacterial infections in various subjects and for treating diseases and conditions that are caused by or mediated by microbial infections. Some embodiments of the invention are directed to methods for treating diseases related to or associated with bacterial infections.

Diseases or conditions that are amenable to treatment with the Lsp-modulating compounds of the invention encompass infections of a subject, particularly a human subject, by any bacteria or other microorganisms that express the Lsp enzyme (e.g., Staphylococcus species or Bacillus species). Specific examples of human diseases caused by or associated with bacterial infections include, e.g., tuberculosis (caused by Mycobacterium tuberculosis), pneumonia (caused by Streptococcus and Pseudomonas), gastritis and ulcers (caused by Helicobacter pylori), foodborne illnesses (caused by bacteria such as E. coli, Shigella, Campylobacter, and Salmonella), gonorrhea (caused by Neisseria gonorrhoeae), meningitis (caused by Neisseria meningitides), tetanus, typhoid fever, diphtheria, syphilis, and leprosy.

The Lsp-inhibiting compounds are useful for treating a subject who is a carrier of any pathogenic bacteria. They can be used to treat a subject who is diagnosed with active bacterial infections. The compounds are also useful in the treatment or prophylaxis of bacterial infection-related conditions in such subjects. Subjects who have not been diagnosed as having a bacterial infection-related disease (e.g., lupus), but are believed to be infected by a pathogenic bacterium and are at risk of developing the disease, are also amenable to treatment with the Lsp-inhibiting compounds of the present invention.

The Lsp inhibitors of the present invention can be directly administered under sterile conditions to the subject to be treated. The compounds can be administered alone or as the active ingredient of a pharmaceutical composition. The therapeutic composition of the present invention can also be combined with or used in association with other therapeutic agents for treating bacterial infections (e.g., other known antibiotics). In some applications, a first Lsp inhibitor is used in combination with a second Lsp inhibitor in order to inhibit bacterial infection to a more extensive degree than cannot be achieved when one Lsp inhibitor is used individually. In some other applications, an Lsp-modulating compound of the present invention may be used in conjunction with known antibiotic agents such as penicillin.

The invention provides pharmaceutical compositions that are derived from the specific Lsp inhibitors described herein or their functional derivatives. Pharmaceutical compositions of the present invention typically comprise at least one Lsp specific inhibitor as the active ingredient. The compositions can optionally also contain one or more acceptable carriers or excipients thereof. In some embodiments, the active ingredient of the pharmaceutical compositions of the invention consists of or consists essentially of an Lsp-inhibiting compound described herein. Pharmaceutically acceptable carriers enhance or stabilize the composition, or facilitate preparation of the composition. Pharmaceutically acceptable carriers are determined in part by the particular composition being administered (e.g., nucleic acid, protein, or small molecules), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, intravenous, or parenteral. For example, the Lsp-inhibiting compound may be complexed with carrier proteins such as ovalbumin or serum albumin prior to their administration in order to enhance stability or pharmacological properties.

The pharmaceutical compositions can be prepared in various forms, such as granules, tablets, pills, suppositories, capsules, and the like. The concentration of therapeutically active compound in the formulation may vary from about 0.1 to 100% by weight. Therapeutic formulations are prepared by any methods well known in the art of pharmacy. The therapeutic formulations can be delivered by any effective means which could be used for treatment. See, e.g., Goodman &Gilman's The Pharmacological Bases of Therapeutics, Hardman et al., eds., McGraw-Hill Professional (10^{th} ed., 2001); Remington: The Science and Practice of Pharmacy, Gennaro (ed.), Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

The therapeutic formulations can be conveniently presented in unit dosage form and administered in a suitable therapeutic dose. A suitable therapeutic dose can be determined by any of the well-known methods such as clinical studies on mammalian species to determine maximum tolerable dose and on normal human subjects to determine safe dosage. Except under certain circumstances when higher dosages may be required, the preferred dosage of an Lsp inhibitory compound usually lies within the range of from about 0.001 to about 1000 mg, more usually from about 0.01 to about 500 mg per day.

The preferred dosage and mode of administration of an Lsp inhibitor can vary for different subjects, depending upon factors that can be individually reviewed by the treating physician, such as the condition or conditions to be treated, the choice of composition to be administered, including the particular Lsp inhibitors, the age, weight, and response of the individual subject, the severity of the subject's symptoms, and the chosen route of administration. As a general rule, the quantity of an Lsp inhibitor administered is the smallest dosage which effectively and reliably prevents or minimizes the conditions of the subjects. Therefore, the above dosage ranges are intended to provide general guidance and support for the teachings herein, but are not intended to limit the scope of the invention.

### EXAMPLES

The following examples are offered to illustrate, but not to limit the present invention.

### Example 1: Expression and Purification of Lipoprotein Signal Peptidases (Lsps)

We developed a strategy for obtaining functional Lsp enzymes that are recombinantly-produced and solubilized. The full length E. coli Lsp clone was generated using colony PCR of E. coli K12 with forward primer CGCCATATGAGTCAATCGATCTGTTCAACAG (SEQ ID NO:1) and reverse primer CGCGGATCCTTATTGTTTTTTCGCTTTAGAAGGTAAAAAACC (SEQ ID NO:2) and verified via double-stranded plasmid sequencing. We then overexpressed Lsp protein as an N-terminal His-tag fusion with a pET19b vector (Agilent) in E. coli BL21(DE3)pLysS competent cells (Agilent). Specifically, cells were grown in 2×YT media supplemented with 100 μg/ml carbenicillin and 35 μg/mIchloramphenicol at 37° C. to an OD600 nm of 0.6. Flasks were then transferred to 16° C., and protein expression was induced with 0.1 mM IPTG overnight. Cells were harvested and resuspended in ice-cold lysis buffer (PBS, 5% v/v glycerol, pH 7.4 supplemented with 1 mg/ml lysozyme, 0.1 mg/ml DNase, 1 mM MgCl₂, 1 mM CaCl₂) and subjected to 2 cycles of lysis by microfluidization (Microfluidics).

To solubilize membrane proteins, n-Dodecyl β-D-maltoside (DDM) was added to give a final concentration of 0.8% w/v, and the lysate was stirred at 4° C. for 2 hours. Elution buffer (PBS, 5% glycerol, 0.1% DDM, 500 mM imidazole, pH 7.4) was added to a final imidazole concentration of 20 mM. The unclarified lysate was then loaded onto a 1 ml HisTrap FF crude Ni-NTA affinity column (GE). The column was pre-equilibrated with wash buffer (PBS, 5% glycerol, 0.1% DDM, 20 mM imidazole, pH 7.4) and eluted with a linear gradient over 20 column volumes. The eluted protein was immediately subjected to gel filtration chromatography (Superdex 200, GE) in PBS, 5% glycerol, 0.1% DDM, pH 7.4. Fractions containing Lsp were supplemented with glycerol to a final concentration of 20%, frozen in liquid N₂ and stored at −80° C. We determined that pure Lsp yields are approximately 1 mg/L of culture with >95% purity, as assessed by SDS-PAGE.

In addition to E. coli Lsp, we also cloned and expressed Lsp from a variety of bacterial species, as the protein is highly conserved among both Gram⁺ and Gram⁻ species. Using the same strategy developed for E. coli Lsp, we have cloned, expressed, and purified Lsp from Streptococcus pyogenes and Thermotoga maritima. The results indicate that all Lsp proteins, regardless of species, can be similarly cloned into a pET vector (e.g., pET19b) and purified as described for E. coli Lsp.

### Example 2. Synthesis of Lsp FRET Substrate

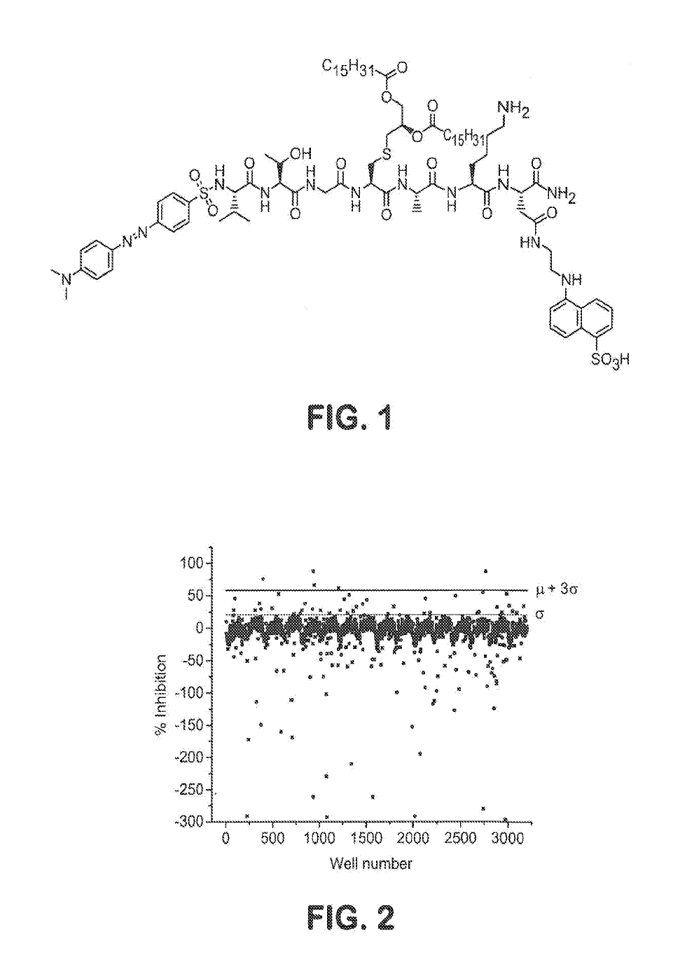

The Lsp FRET peptide substrate sequence Dabsyl-VTGC((R)-2,3-di(palmitoyloxy)-propyl)AKD(EDANS) (FIG. 1) was based on the lipobox region of a putative acid phosphatase from Streptococcus pyogenes (NCBI Reference Sequence: NP 269874). This sequence was chosen to afford maximum signal-to-background in an assay with recombinant purified E. coli Lsp from a library of lipoprotein mimetic peptides of varying length based on known lipoprotein sequences or permutations of common lipobox residues. The peptide was synthesized using standard Fmoc solid phase synthesis chemistry on NovaSyn TGR resin (EMD Millipore), using FRET fluorescence donor and acceptor pair EDANS/Dabsyl. Specifically, Fmoc-(R)-Cys((R)-2,3-di(palmitoyloxy)-propyl)-OH was prepared as a pure diastereomer according to Hida et al., J. Antibiot. (Tokyo) 48, 589-603, 1995. Peptide couplings were performed using 3 equivalents Fmoc-amino acid, 3 equivalents benzotriazol-1-yl-oxytripyrrolidinophosphonium hexafluorophosphate (PyBOP), and 6 equivalents diisopropylethylamine (DIPEA) in dimethylformamide (DMF) for 1 hour at room temperature, and deprotections were performed using 20% v/v pyrrolidine in DMF for 15 minutes. EDANS was incorporated via Fmoc-Asp(EDANS)-OH and Dabsyl via reaction with 3 equivalents Dabsyl chloride and 6 equivalents DIPEA overnight.

After completion of the peptide synthesis, the substrate was released from the resin with a cocktail of trifluoroacetic acid, triisopropylsilane, and water (95%, 2.5%, 2.5% v/v/v) for 2 hours at room temperature. Crude substrate was purified by normal-phase HPLC using an XBridge Amide column (Waters) and methanol/dichloromethane mobile phase with a linear gradient of 15-100% methanol. The final purity of Dabsyl-VTGC((R)-2,3-di(palmitoyloxy)-propyl)AKD(EDANS) exceeded 95% (HPLC) and was verified by mass spectrometry: expected m/z 1776.96, LC/MS (ESI) m/z 1777.97 (M+H—) and 889.99 (M+2H—). Michaelis-Menten kinetic measurements for the hydrolysis of the peptide by 400 nM of purified Lsp in assay buffer (PBS, 5% glycerol, 0.5% DDM pH 7.4) yielded a K_{M}=5 μM with apparent substrate inhibition evident above 100 μM.

In the FRET substrate for assaying Lsp activity, we can substitute the specific peptide with any peptide sequence that contains a lapidated Cys residue. For example, we have made FRET substrates based on Braun's lipoprotein as well as several different lengths and amino acid substitutions of the specific peptide example in FIG. 1.

### Example 3. High Throughput Screening for Lsp Inhibitors

Utilizing the purified and solubilized Lsp enzyme, we developed an in vitro high-throughput functional screening for the integral membrane protease Lsp. First, screening of the Maybridge HitFinder library (Thermo Fisher) was conducted in black polystyrene 384-well low volume plates (Greiner #788076) using a BioRAPTR reagent dispenser (Beckman Coulter). Purified Lsp was diluted to 500 nM in assay buffer (PBS, 5% glycerol, 0.5% DDM pH 7.4) and 104 of this stock was added into each well. Next, 100 nL of a 2 mM stock of library compounds were pinned into each well and plates were centrifuged briefly. Following incubation at room temperature for 30 minutes, 2.5 μL of 250 μM Lsp FRET substrate in 25% dimethyl sulfoxide (DMSO), 75% assay buffer was added to each well. The plates were spun down and incubated for 30 minutes at room temperature. The assay was quenched with 2.5 μL of a solution containing 500 mM zinc chloride in water, giving a final concentration of 83 mM. The concentration of Lsp, FRET substrate, and library compounds during the activity assay were 400 nM, 50 μM, and 16 μM, respectively. The final DMSO concentration in the assay is 5%, where maximal Lsp activity occurs in 5-10% DMSO.

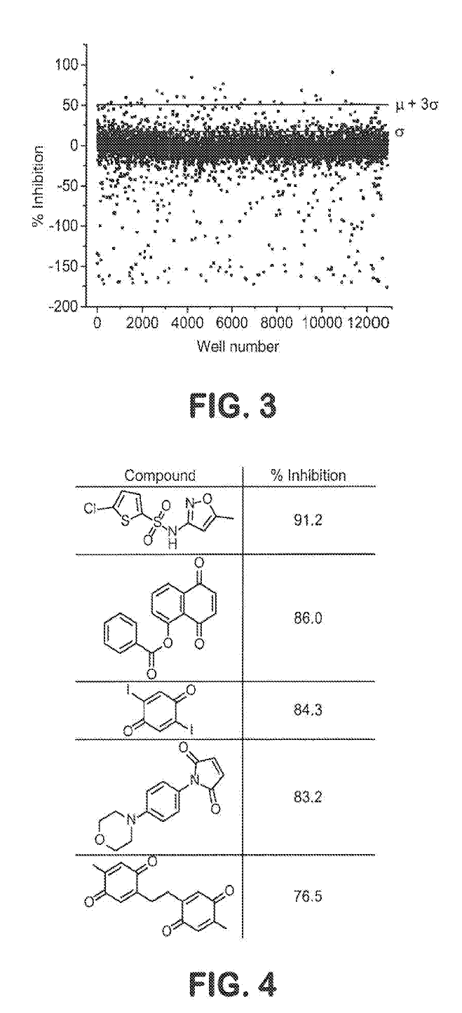

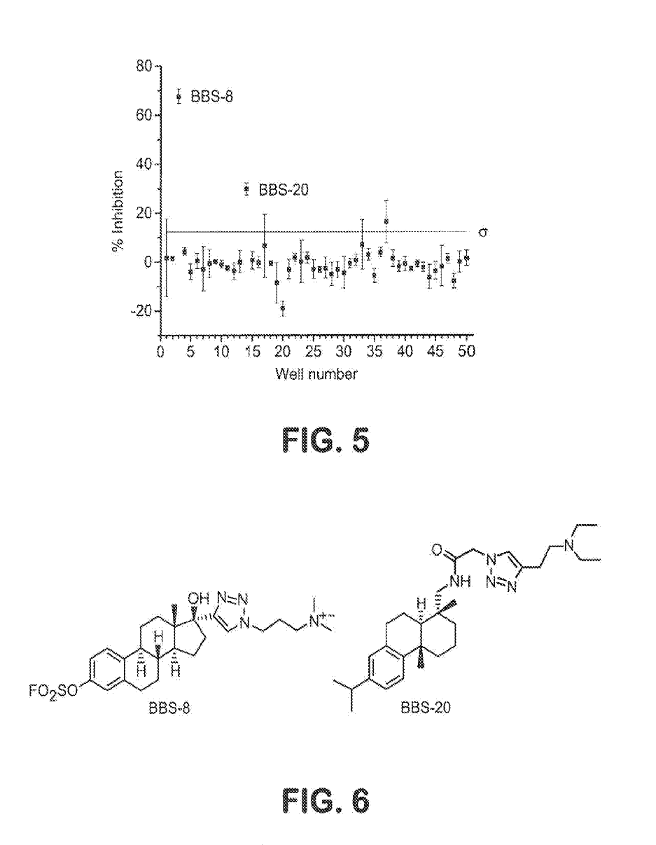

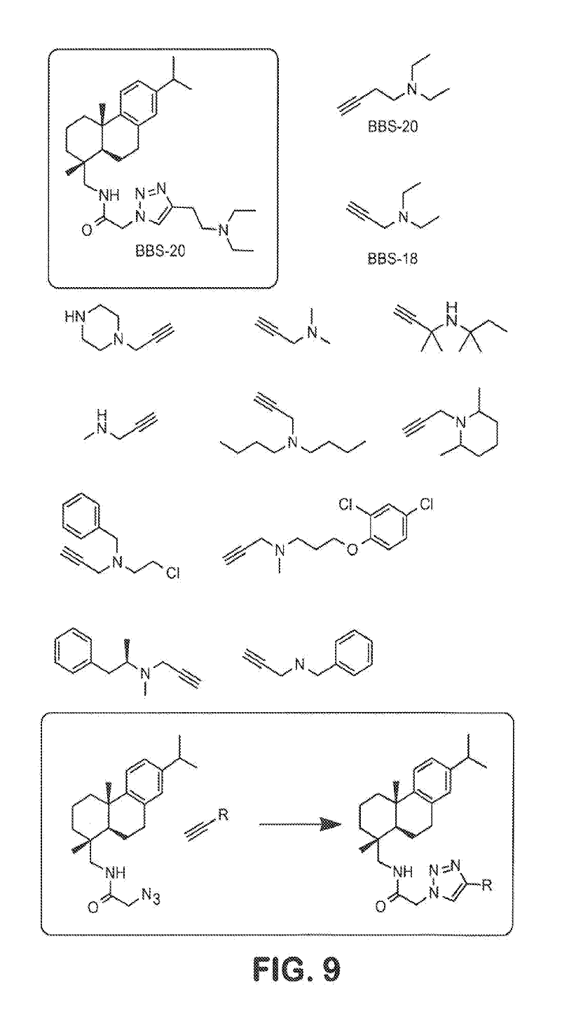

Catalytic signal was defined as the full enzyme activity pinning DMSO only, and background signal was measured by adding substrate to assay buffer containing no Lsp. An initial screen of 3,200 compounds (FIG. 2) yielded a Z′=0.79, signal-to-background ratio (S/B)=2.1, mean % inhibition (μ)=−4.32, standard deviation of % inhibition (σ)=20.89. For a cutoff of μ+3σ, the hit rate was 0.15%. A larger screen of 12,800 compounds (FIG. 3) yielded a Z′=0.74, S/B=2.0, μ=−3.66, α=16.90. For a cutoff of μ+3σ, the hit rate was 0.3%. The hits with the highest % inhibition primarily consisted of compounds with known nonspecific reactivity or pan-assay interference (FIG. 4. In addition, screening of Sharpless laboratory compounds (FIG. 5) was performed identically, except that each compound was present in triplicate on the screening plate, and yielded a Z′=0.86 and S/B=2.2.

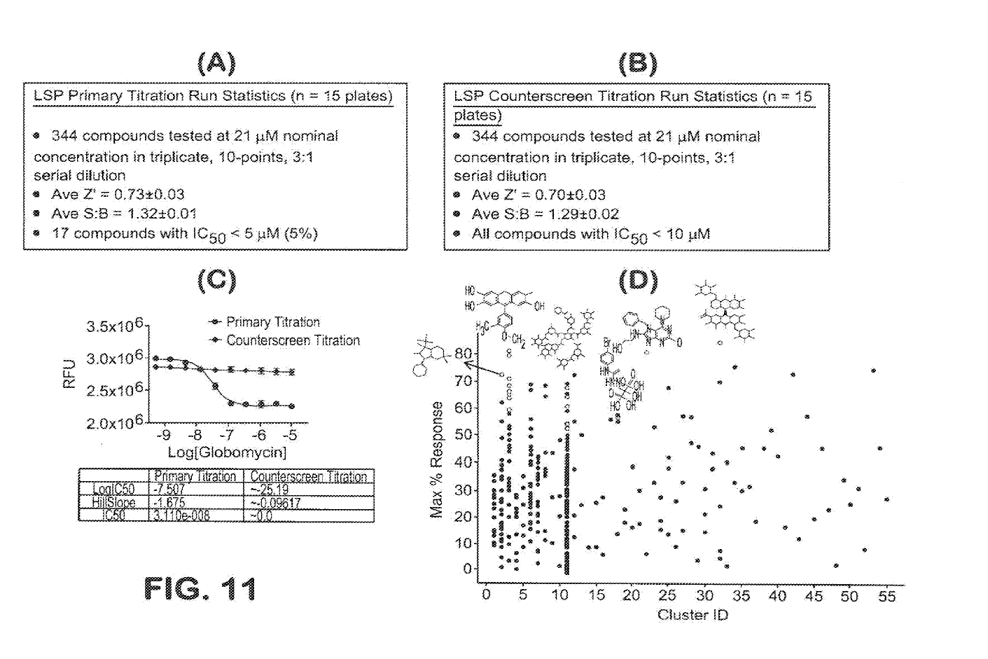

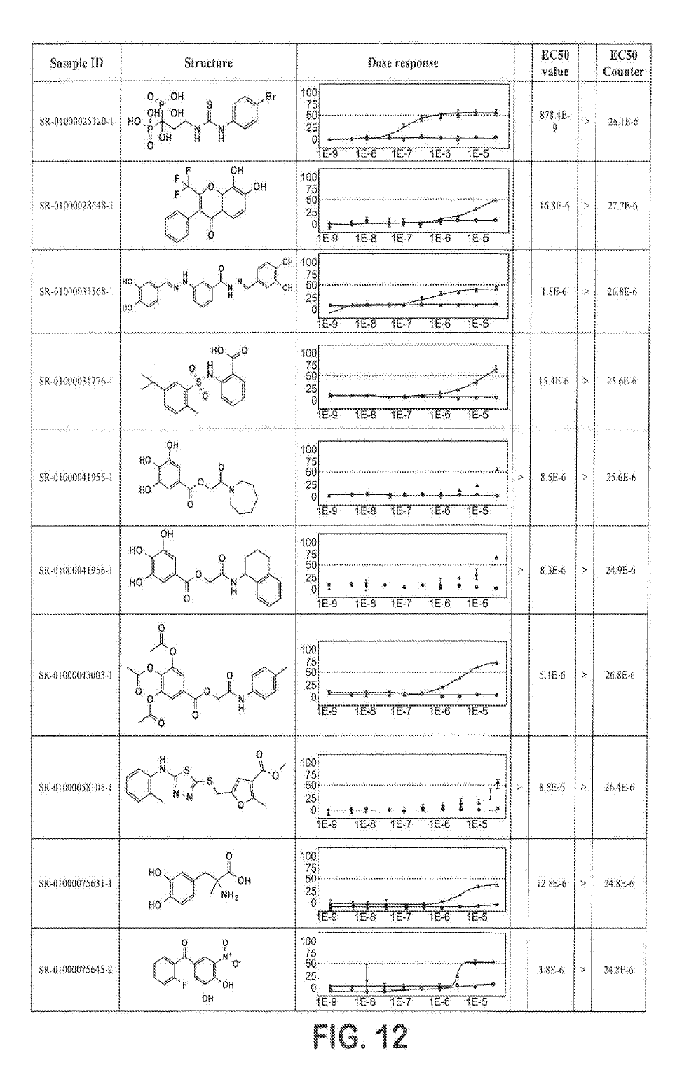

From the Sharpless library, we identified two inhibitors based on terpenoid natural products: BBS-8, an estradiol derivative with a fluorosulfonate warhead installed on the A ring hydroxyl group, and BBS-20, a leelamine derivative (FIG. 6). We validated the inhibition of varying concentrations of BBS-20 against 400 nM purified Lsp and calculated an IC₅₀ of 15±4 μM (FIG. 7). We tested several fixed concentrations of BBS-20 against varying concentrations of Lsp FRET substrate to determine a mechanism of inhibition (FIG. 8). Increasing concentrations of BBS-20 reduced the enzyme's maximum initial velocity but not the apparent K_{M} within error, indicating a noncompetitive mode of inhibition similar to the peptide Lsp inhibitor globomycin.

All hits can be revalidated with our in vitro FRET substrate cleavage assay, as shown for BBS-20. In addition, a secondary in vitro validation assay can be performed. This assay involves the detection of substrate cleavage by gel filtration size exclusion chromatography and/or HPLC. Binding kinetics of Lsp with small molecule inhibitors can be measured by surface plasmon resonance (SPR) as well as the heat of release due to the binding event with isothermal calorimetry (ITC). Crystal structures of Lsp from a variety of bacterial species can also be examined, which would aid in the discovery and advancement of all inhibitors. Further, small molecules of interest that pass the in vitro validation assays can be subjected to kill curve assays utilizing a panel of Gram⁻ and Gram′ bacteria to determine efficacy of use as a bactericidal agent.

### Example 4. Ultra-High Throughput Screening and Validation Assays for Lsp Inhibitors

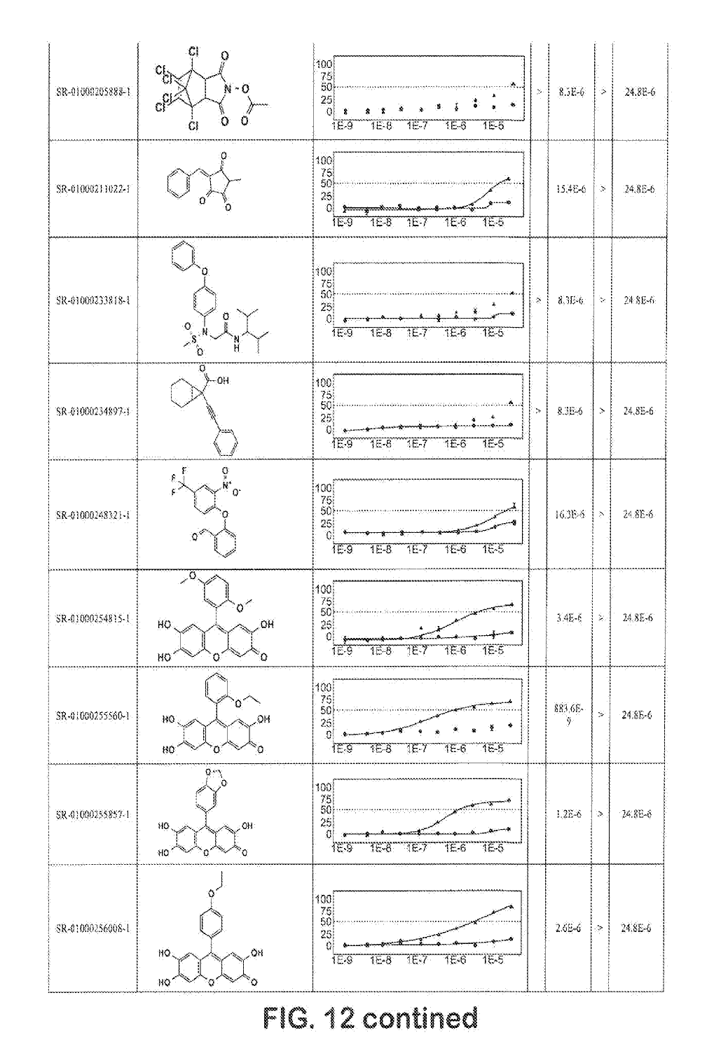

We further optimized and miniaturized our in vitro high-throughput functional screening for the integral membrane protease Lsp into 1,536-well format. The primary screen of 646,275 Scripps Drug Discovery Library (SDDL) compounds was conducted at The Scripps Research Institute, Florida (TSRI FL). Purified Lsp was diluted to 200 nM in assay buffer (PBS, 5% glycerol, 10 mM DTT, 0.5% DDM pH 7.4) and 4 μL of this stock was added into each well. Next, 50 nL of stock TSRI FL library compounds were pinned into each well to a final concentration of 8.4 μM and plates were centrifuged briefly. Following incubation at room temperature for 30 minutes, 14 of Lsp FRET substrate in 25% dimethyl sulfoxide (DMSO), 75% assay buffer was added to each well to a final substrate concentration of 20 The plates were spun down and incubated for 60 minutes at room temperature. The assay was quenched with 1 μL of a solution containing 500 mM zinc chloride in water, giving a final concentration of 83 mM. The final DMSO concentration in the assay is 5%, where maximal Lsp activity occurs in 5-10% DMSO (FIG. 10). Fluorescence was measured on an EnVision pate reader (ex. 355 nm; em. 495 nm).

Raw fluorescence assay data was imported into TSRI corporate database and subsequently analyzed using Symyx software. Activity of each compound was calculated on a per-plate basis using the following equation:

$$Percent Response of compound = 100 \times ( \frac{Median Low Control - Test Well}{Median Low Control - Median High Control} )$$

Where “High Control” represents wells containing No LSP+DMSO and “Low Control” represents wells containing LSP+DMSO and “Data Wells” contain LSP+test compound. The Z′ and signal-to-background ratio (S/B) for this assay is calculated using the High Control and Low Control wells. The screen of 645,275 SDDL compounds yielded a Z′=0.69±0.05, and S/B of 1.35±0.05 (n=519 plates). Using an “Interval Cutoff” (=27.92% inhibition) the primary assay yielded 2,271 active compounds (“hits”)

A confirmation screen used the same reagents and detection system as the primary screening assay, but tested each of the 2,271 compounds at a single concentration (nominally 8.43 μM) in triplicate. The pre-quench counterscreen assay was similar in format to the LSP primary assay and employed the same reagents and the same readout but, pinned the compounds after quenching the enzymatic reaction. The “High Control” for this counterscreen assay was also No LSP+DMSO. The “Low Control” was LSP+DMSO. This assay was used to identify sundry “off-target” hits that affected fluorescence measurement, such as fluorescent quenchers.

The LSP confirmation assay performance was excellent with an average Z′ of 0.74±0.02 and a S/B of 1.41±0.01. Using the assay cut-off of 27.92% response (Primary Cutoff), 698 hits confirmed with activity greater than 27.92%. The pre-quench counterscreen assay performance was also excellent with an average Z′ of 0.73±0.03 and a S/B of 1.38±0.01. Using the same cutoff as the confirmation assay, 455 hits were found. Of the 2,271 compounds tested, 698 compounds confirmed activity in the LSP primary assay, and 344 of these demonstrated selective activity, i.e. they were inactive in the pre-quench counterscreen assay.

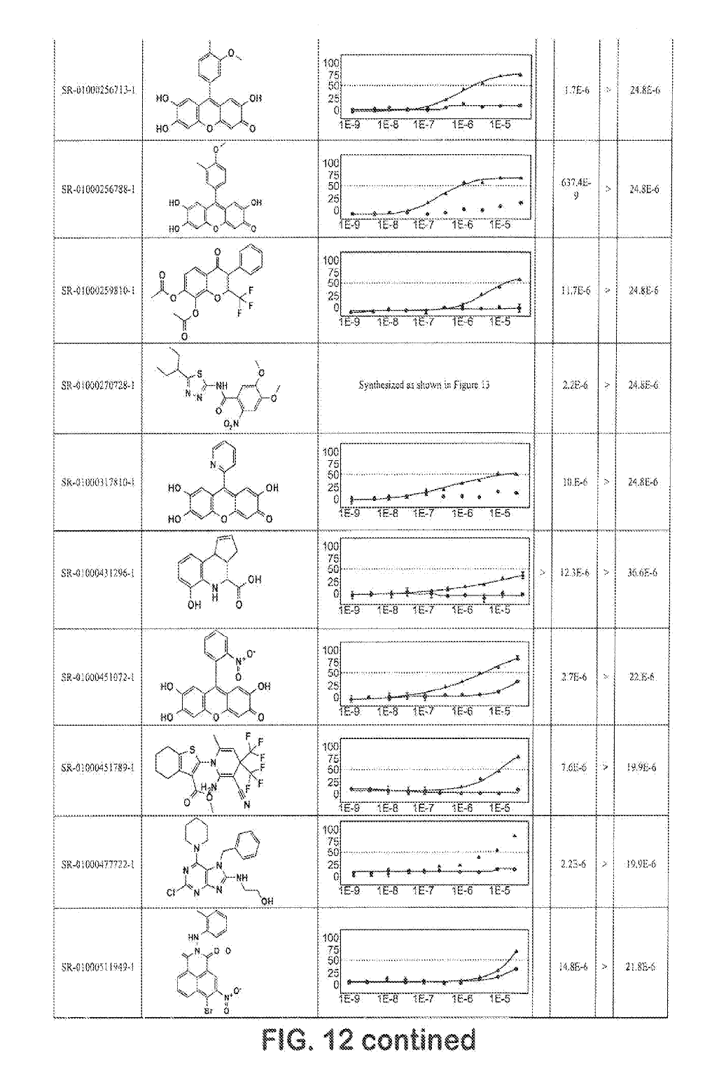

The 344 compounds were subjected to a dose-dependent titration assay with 10-point dose-response titrations (3-fold dilutions) in triplicate. LSP primary and pre-quench titration assays employed the same reagents, protocols, and detection systems as the secondary assays. The LSP primary titration assay performance was excellent with an average Z′ of 0.73±0.03 and a S/B of 1.32±0.01. The LSP pre-quench counterscreen titration assay performance was also excellent with an average Z′ of 0.70±0.03 and a S/B of 1.29±0.02 (FIG. 11). Globomycin was included in the both titration assays as a control. For each test compound, percent activity was plotted against compound concentration. A four parameter equation describing a sigmoidal dose-response curve was then fitted with adjustable baseline using Assay Explorer software (Symyx Technologies Inc.). The reported IC₅₀ values were generated from fitted curves by solving for the X-intercept value at the 50% activity level of the Y-intercept value. Representative compounds and the dose response curves with error bars from triplicate experiments are show in FIG. 12.

We synthesized SR-01000270728-1 from starting materials as shown in FIG. 13. An in vitro dose-response assay was performed on E. coli Lsp and shown to inhibit with an EC50 of 2.2 μM. Similar synthesis and validation assays can be readily performed for the other compounds in FIG. 12.

E. coli Lsp coding sequence (Accession No.
CAQ30547; SEQ ID NO: 3):
ATGGGCCATCATCATCATCATCATCATCATCATCACAGCAGCGGCCAT
ATCGACGACGACGACAAGCATATGAGTCAATCGATCTGTTCAACAGGGCT
ACGCTGGCTGTGGCTGGTGGTAGTCGTGCTGATTATCGATCTGGGCAGCA
AATACCTGATCCTCCAGAACTTTGCTCTGGGGGATACGGTCCCGCTGTTC
CCGTCGCTTAATCTGCATTATGCGCGTAACTATGGCGCGGCGTTTAGTTT
CCTTGCCGATAGCGGCGGCTGGCAGCGTTGGTTCTTTGCCGGTATTGCGA
TTGGTATTAGCGTGATCCTGGCAGTGATGATGTATCGCTCGAAGGCCACG
CAGAAGCTAAACAATATCGCTTACGCGCTGATTATTGGCGGCGCGCTGGG
CAACCTGTTCGACCGCCTGTGGCACGGCTTCGTTGTCGATATGATCGACT
TCTACGTCGGCGGCTGGCACTTCGCCACCTTCAACCTTGCCGATACTGCC
ATCTGTGTCGGTGCGGCACTGATTGTGCTGGAAGGTTTTTTACCTTCTAA
AGCGAAAAAACAATAA.
S. pyogenes Lsp coding sequence (Accession No.
AAK33759; SEQ ID NO: 4):
ATGAAAAAACGATTGTTTGTGCTTAGCTTGATCCTCCTTGTAGCTTTG
GATCAACTTAGTAAATTTTGGATTGTTTCTCATATAGCGCTTGGAGAAGT
GAAACCCTTTATCCCAGGTATCGTCAGCTTGACTTACTTGCAAAACAATG
GGGCTGCCTTTTCCATATTGCAGGACCAGCAATGGTTCTTTGTTGTCATA
ACGGTTTTAGTTATCGGTTATGCTATTTATTACCTTGCTACTCATCCCCA
TTTAAATATCTGGAAACAATTAGCTCTCTTGCTTATTATTTCTGGTGGAA
TCGGGAATTTTATTGATCGTTTGCGTTTAGCTTACGTGATTGATATGATT
CATTTAGACTTTGTGGATTTTGCCATTTTTAATGTGGCAGATTCATACCT
TACCGTTGGTGTCATATTATTATTGATATGTTTATGGAAAGAAGAGGATT
ATGGAAATCTCGAGCACCACCACCACCACCACTGA.
T. maritima Lsp coding sequence (Accession No.
NP_228273; SEQ ID NO: 5):
ATGGGCCATCATCATCATCATCATCATCATCATCACAGCAGCGGCCAT
ATCGACGACGACGACAAGCATATGGCGTTTGTGATGGTTCTCACAATTGT
TCTGGATCAGCTTACAAAGCGGATAGCAAGCGAGATACACGGAACTTTTT
TCATAGTTCCGGGTTTTTTGAGATTCGTGAAGGCAACCAACCGAGGAATC
GCACTCGGGTTGTTTAAAAATCTTTCCGAACAGCTTCTCTGGACCGTGAT
GTTCGTTGTTGTTTTTCTCTCCCTGCTTCCTTATATTTTCAAGTTCAGCA
GGCTGGAAAGAATAGCCATGGGCTTCATTCTTGGGGGAGCTCTCGGCAAC
CTTCTCGACAGAATCAGATTCGGATACGTTCTTGATTTTCTGAACTTGAC
CTTTCTCCCAACGATATTCAACCTAGCGGATGTGTTCATCATAGTCGGAG
GAGCGCTTATGATACTGGGAGTTTTCAGAGGTGGAGACAATGAAAGTTTG
GAGAGTCGAAAAAAGAGAAGAGGGCTGGAGACTGGATCAGTTTTTGAAGG
AGAAGACACCATCATGGATCTCGAGATCAATGATTCAAAAAGCGATAAAA
GAGGGCAAAGTGAAGGTCAACGGTCAGATTAA.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

What is claimed is:

1. An assay system for measuring catalytic activity of a lipoprotein signal peptidase (Lsp), comprising (a) a recombinantly-expressed, soluble and purified Lsp enzyme and (b) an Lsp substrate.

2. The assay system of claim 1, wherein the Lsp is a bacterial Lsp.

3. The assay system of claim 2, wherein the Lsp is E. coli Lsp.

4. The assay system of claim 1, wherein the Lsp is expressed as a His-tagged fusion protein.

5. The assay system of claim 4, wherein the His-tagged fusion protein comprises an N-terminal His₆-tag.

6. The assay system of claim 1, wherein the Lsp is solubilized with a detergent.

7. The assay system of claim 6, wherein the detergent is n-Dodecyl β-D-maltoside (DDM).

8. The assay system of claim 1, wherein the substrate is a peptide, a peptide mimetic, or a protein that contains a lipid-modified cysteine residue.

9. The assay system of claim 1, wherein the substrate is labeled with a fluorescence resonance energy transfer (FRET) donor-acceptor pair.

10. A method for identifying agents that inhibit a lipoprotein signal peptidase (Lsp), comprising (a) contacting a recombinantly-produced and purified Lsp with a Lsp substrate in the presence of test compounds, and (b) detecting inhibition by one or more test compounds of Lsp cleavage of the substrate; thereby identifying agents that inhibit the lipoprotein signal peptidase (Lsp).

11. The method of claim 10, wherein the Lsp is a bacterial Lsp.

12. The method of claim 11, wherein the Lsp is E. coli Lsp.

13. The method of claim 10, wherein the Lsp is a His-tagged fusion protein.

14. The method of claim 14, wherein the His-tagged fusion protein comprises an N-terminal His₆-tag.

15. The method of claim 10, wherein the Lsp is solubilized with a detergent.

16. The method of claim 16, wherein the detergent is n-Dodecyl β-D-maltoside (DDM).

17. The method of claim 10, wherein the substrate is a peptide, a peptide mimetic, or a protein that contains a lipid-modified cysteine residue.

18. The method of claim 10, wherein the substrate is labeled with a fluorescence resonance energy transfer (FRET) donor-acceptor pair.

19. The method of claim 10, wherein Lsp catalytic activity is detected via fluorescence resonance energy transfer.

20. The method of claim 10, which is performed in a high throughput format.

21. The method of claim 10, wherein the test compounds are small organic compounds.

22. The method of claim 10, further comprising examining the identified agents for bactericidal activity.

23. The method of claim 10, further comprising examining the identified agents for ability to inhibit bacterial lipoprotein diacylglyceryl transferase (Lgt).

24. A method for inhibiting Lsp catalytic activity in a bacterial cell, comprising contacting the bacterial cell with an Lsp inhibitor compound under conditions to allow the compound to inhibit Lsp that is present in the cell, wherein the Lsp inhibitor compound is Compound SR-010000270728-1, Compound BBS-8 or Compound BBS-20, or a functional variant thereof.

25. The method of claim 24, wherein the bacterial cell in present inside a subject.

26. The method of claim 25, wherein the subject is afflicted with an infection by the bacterial cell.

27. The method of claim 25, wherein the subject is administered a therapeutically effective amount of the Lsp inhibitor compound.

28. A method for inhibiting bacterial growth and treating bacterial infection in a subject, comprising administering to the subject afflicted with a bacterial infection a pharmaceutical composition comprising a therapeutically effective amount of an Lsp inhibitor compound, thereby inhibiting bacterial growth and treating bacterial infection in the subject; wherein the Lsp inhibitor compound is Compound SR-010000270728-1, Compound BBS-8 or Compound BBS-20, or a functional variant thereof.

29. The method of claim 28, wherein the subject is a human.

30. A method of generating an active detergent-solubilized transmembrane enzyme capable of measuring catalytic activity in an assay system, comprising (a) constructing an expression vector capable of expressing the active transmembrane enzyme; (b) expressing said active transmembrane enzyme from said vector; and (c) solubilizing and purifying the active transmembrane enzyme in a detergent based system; thereby generating an active transmembrane detergent-solubilized enzyme capable of measuring specific catalytic activity in an assay system.

31. The method of claim 30, wherein the transmembrane enzyme is Lsp.

32. Use of the transmembrane enzyme produced according to claim 30 in an assay to measure catalytic activity of the enzyme.

33. Use of the transmembrane enzyme produced according to claim 30 in a high throughput screen to identify specific inhibitors of the transmembrane enzyme.

34. Use of the transmembrane enzyme produced according to claim 33, wherein the transmembrane enzyme is Lsp.

35. A method for identifying an Lsp-inhibitory compound with improved properties, comprising (a) synthesizing one or more structural analogs of a lead Lsp inhibitor compound; (b) performing a functional assay on the analogs to identify an analog that has an improved biological or pharmaceutical property relative to that of the lead compound; thereby identifying an Lsp-inhibitory compound with improved properties.

36. The method of claim 35, wherein the lead Lsp inhibitor compound is Compound SR-010000270728-1, Compound BBS-8, Compound BBS-20, or a functional variant thereof.

37. The method of claim 35, wherein the improved biological or pharmaceutical property is an enhanced inhibition of Lsp catalytic activity.

38. The method of claim 37, wherein the functional assay utilizes a purified and detergent-solubilized Lsp enzyme.

39. The method of claim 35, wherein the improved biological or pharmaceutical property is an increased stability or serum half-life.
